(19) United States
(12) Patent Application Publication
(10) Pub. No.: US 2026/0191672 A1
(43) Pub. Date: Jul. 9, 2026

(54) Foot Supination Control Device

(71) Applicant: ReallyHadToMove Industries, LLC

(72) Inventor: Jon Erdmann, Sacramento, CA (US)

(21) Appl. No.: 19/438,222

(22) Filed: Dec. 31, 2025

Related U.S. Application Data
(60) Provisional application No. 63/741,550, filed on Jan. 3, 2025.

(51) Int. Cl. A61F 5/01; A61F 5/30

(52) U.S. Cl. CPC A61F 5/0113; A61F 5/30

(57) ABSTRACT

An open sided foot supination control device has a base plate having a first end and an opposing second end, and a talus bone pressure applicator having a first end and an opposing second end. A lateral member having a first end and second end is joined to the first end of the base plate, and an opposite end of the lateral member is joined to the first end of the talus bone pressure applicator. The foot supination control device is formed to have the base plate under the person's foot, while the talus bone pressure applicator is positioned to provide supination preventing pressure to the talus bone.

### CROSS-REFERENCE TO RELATED APPLICATION(S)

This application claims the benefit and priority of U.S. Provisional Patent Application No. 63/741,550 , titled “Supination Control Device,” filed Jan. 3, 2025, the contents of which are hereby incorporated by reference in its entirety.

### FIELD

This subject matter relates to an Ankle Foot Orthosis (AFO) that addresses drop foot with a focus on prohibiting supination/inversion of the foot. Specifically, a unitary like device which provides pressure on the talus bone thereby preventing the rise of the talus bone which often, when unprevented, results in supination.

### BACKGROUND

Supination problems may come or arise from a number of reasons: neurological conditions, biomechanical conditions, talus bone injury, overly high arches, gait deformity, ligament issues, and so forth. For example, supination/drop foot is a condition wherein it is difficult for a person to flex their foot. Existing solutions to supination control include using a Velcro^{tm} (hook and loop fasteners) strap across the ankle. The problem with the hook and loop straps is it essentially locks the ankle in position resulting in immobility of the Range of Motion (ROM) of the ankle. This further negatively impacts many of the subphases of gait, resulting in gait compensation thereby affecting the whole chain of body i.e., Knees, hips, back, etc. Specifically, the patient can injure other parts of his/her body because of the immobility being strap-enforced upon the ankle.

Therefore, there has been a long-standing need in the Orthotics field for a new form of brace(s) or equivalent that eliminates the above issues. Examples of such a new brace(s) is detailed below.

### SUMMARY

The following presents a simplified summary in order to provide a basic understanding of some aspects of the claimed subject matter. This summary is not an extensive overview and is not intended to identify key/critical elements or to delineate the scope of the claimed subject matter. Its purpose is to present some concepts in a simplified form as a prelude to the more detailed description that is presented later.

In one aspect of the disclosed embodiments, an open sided foot supination control device is provided comprising, a base plate having a first end and an opposing second end; a talus bone pressure applicator having a first end and an opposing second end; and a lateral member having a first end and second end, a first end of the lateral member joined to the first end of the base plate, and a second end of the lateral member joined to the first end of the talus bone pressure applicator.

In another aspect of the disclosed embodiments, the above device is provided, wherein the base plate, talus bone pressure applicator and lateral member are unitary, coplanar to each other and formed from a sheet of material, the base plate being larger than the talus bone pressure applicator and the lateral member forming a curved connecting structure between the base plate and the talus bone pressure applicator; and/or wherein the base plate, talus bone pressure applicator and lateral member are in an operational form, wherein the base plate is configured to be disposed on a bottom of a foot and the talus bone pressure applicator is configured to be disposed adjacent to a talus bone of the foot; and/or further comprising a cushion disposed on an underside of the talus bone pressure applicator; and/or wherein at least one of the talus bone pressure applicator and cushion is configured to actively apply pressure to a talus bone of a foot; and/or wherein a shape of the talus bone pressure applicator is curved; and/or wherein an overall shape of the foot supination control device appears to represent a number “2”; and/or wherein a thickness of the sheet of material is between 1/32 to ¼ inches; and/or wherein the sheet of material is composed of at least one of a carbon fiber, polypropylene, nylon, and plastic; and/or wherein the sheet of material becomes temporarily moldable upon an application of heat; and/or wherein the sheet of material becomes hardened upon an application of at least one of a resin and UV light; and/or wherein a height of the supination control device is between 3-6 inches.

In yet another aspect of the disclosed embodiments, a foot supination control device is provided comprising, a base plate having a first end and an opposing second end, the base plate configured to configured to be disposed on a bottom of a foot; a talus bone pressure applicator having a first end and an opposing second end, disposed above the base plate and having a size smaller than the base plate; and a lateral member having a first end and second end, a first end of the lateral member joined to the first end of the base plate, and a second end of the lateral member joined to the first end of the talus bone pressure applicator, wherein the lateral member is configured to rise from the base plate to the talus bone pressure applicator and conform to an outer shape of the foot, wherein the talus bone pressure applicator is configured to actively apply pressure to a talus bone of the foot and the foot supination control device provides unrestricted gait motion.

In yet another aspect of the disclosed embodiments, the above device is provided, wherein the base plate, talus bone pressure applicator and lateral member are unitary and formed from a sheet of material, the base plate being larger than the talus bone pressure applicator and the lateral member forming a curved connecting structure between the base plate and the talus bone pressure applicator; and/or further comprising a talus cushion disposed on an underside of the talus bone pressure applicator; and/or further comprising an Ankle Foot Orthosis (AFO) device, the supination control device being coupled to the AFO; and/or wherein the applied pressure is between 2-3 lbs.

In another aspect of the disclosed embodiments, a method of fitting a supination control device to a foot of a user is provided, comprising, measuring a size of a user's foot; gathering a rigid substrate material, having a base plate, a talus bone pressure applicator and a lateral member, corresponding to the measured foot size; altering the rigid substrate material into a softened state by at least one of heating and applying a softening agent to the substrate material; fitting a protective sock over the user's foot; placing the base plate under the user's foot and bending the lateral member and talus bone pressure applicator to position the talus bone pressure applicator adjacent to a talus bone of the user, while the substrate material is in the softened state; obtaining a talus bone pressure of between 2-3 lbs via at least one of pressing the talus bone pressure applicator and securing a pressure-inducing cushion to the talus bone pressure applicator; and hardening the softened substrate material into a rigid state by at least one of cooling, chemical treatment and UV light exposure to the softened substrate material.

In yet another aspect of the disclosed embodiments, the above method is provided, further comprising, testing a fit of the supination control device; and/or wherein the protective sock is a thermally resistant sock; and/or further comprising forming a first planar state of the supination control device from a sheet of material, by at least one of milling, cutting, molding, and 3-D printing the sheet of material to form the base plate, a talus bone pressure applicator and a lateral member; and/or wherein a shape of the first planar state of the supination control device has an appearance of a numeral “2.”

### BRIEF DESCRIPTION OF THE DRAWINGS

FIG. 1 are anatomy-views of a foot having a talus/supination problem.

FIG. 2A an edge view of a cassette material suitable for forming a Supination Control Device (SCD).

FIG. 2B a top view illustration of a cassette material having a form factor for SCD use.

FIG. 2C a top view illustration of a cassette material having another form factor for SCD use.

FIG. 3 is an illustration of a fitted SCD to a foot.

FIG. 4 is an illustration of an interior view of a SCD.

FIG. 5 is an illustration of another view of a SCD with a cushion.

FIG. 6 is an illustration of side perspective view of a SCD.

FIG. 7 is an illustration of a frontal view of a SCD.

FIG. 8 is an illustration of a frontal perspective view of a SCD.

FIG. 9 is an illustration of a bottom view of a SCD.

FIG. 10 is an illustration of a side perspective view of a SCD worn with an AFO.

FIG. 11 is an illustration of a frontal top view of a SCD worn with an AFO.

FIG. 12 is an illustration of a side view of a SCD worn with an AFO.

FIG. 13 is flow chart of an exemplary process of making and fitting an SCD.

### DETAILED DESCRIPTION

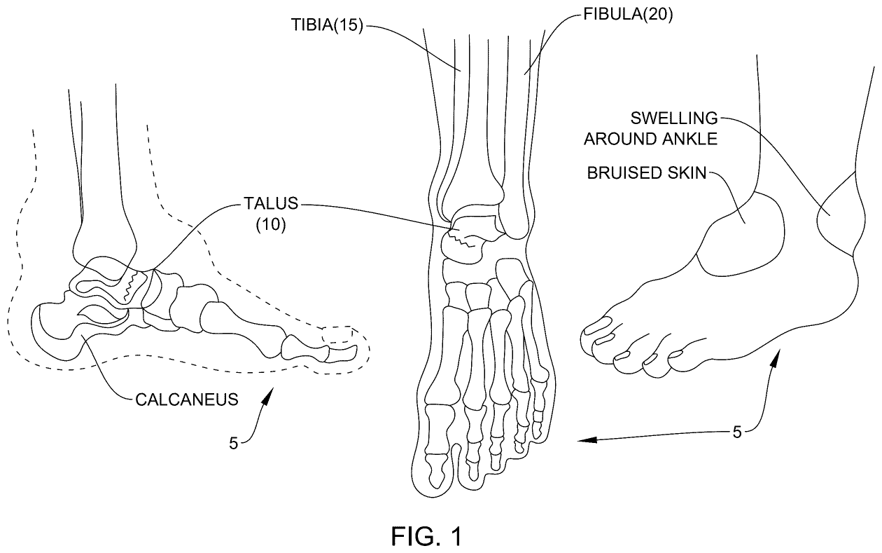

FIG. 1 contains various foot views 5, showing a side “bone” view, a top bone view, and a top perspective view of a person's foot 5 with a talus bone 10 fracture. This FIG. provides the anatomical location of the talus bone 5, wherein the rightmost top perspective view illustrates typical swelling associated with talus bone 5 injury. Very evident is the awkward location of the talus bone 5, wherein conventional attempts to “secure” the talus bone 5 exacerbates the rest of the foot bones, muscles and severely affects the mobility of the person. A better approach to this problem is hereafter described.

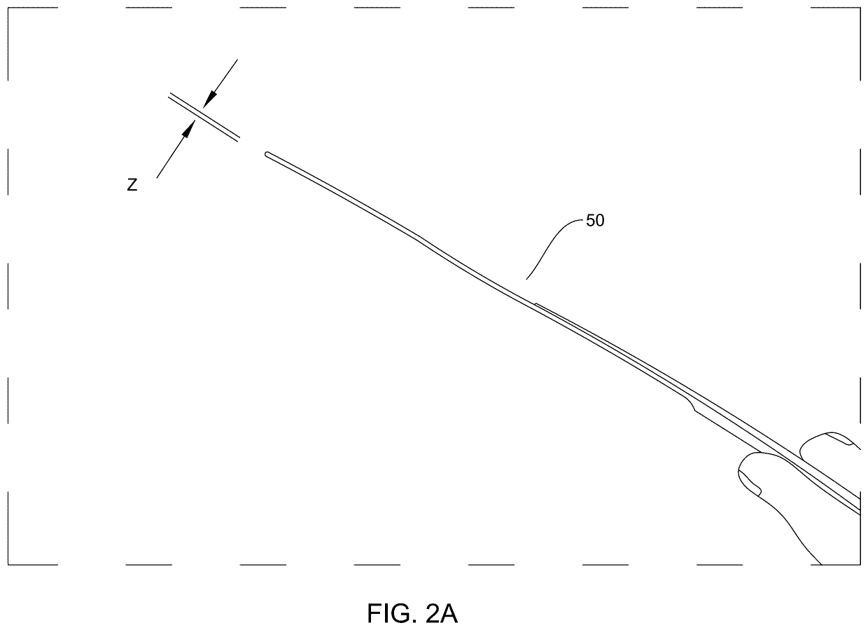

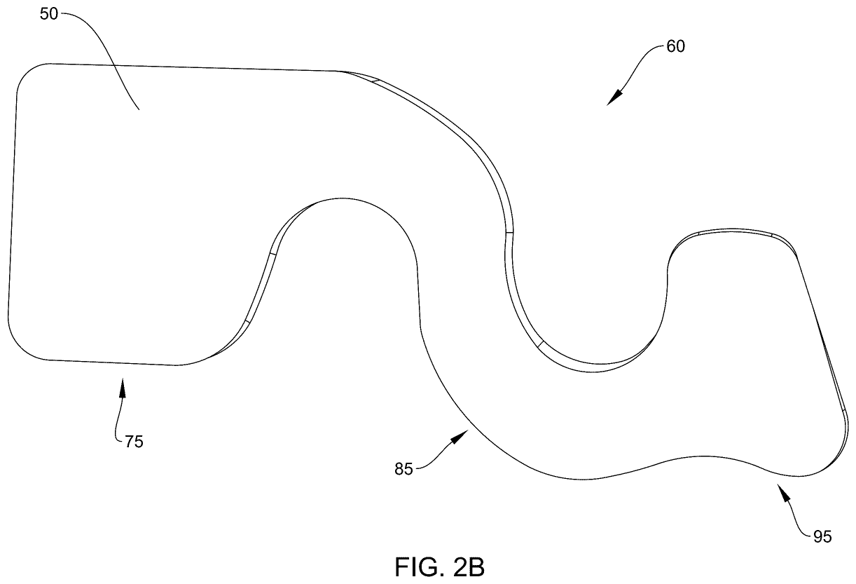

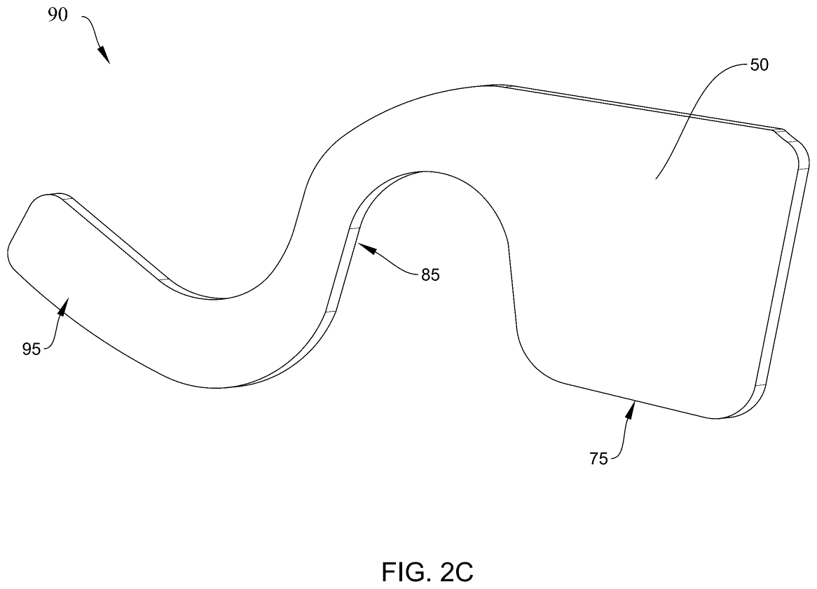

FIG. 2A is a side view of an exemplary cassette material 50, suitable for forming the main body of the supination control device (SCD) described in the following FIGS. The cassette material 50 may be unitary and comprised from a sheet of layered carbon braid, carbon polypropylene braid, or carbon graphite material, resins, or an equivalent (such as nylon, plastic, etc.). The thickness “Z” may vary according to the strength and characteristics of the cassette material 50 and may vary along the material. The SCD can be made in a plethora number of sizes so the cassette material 50 may be an appropriately sized “blank.” Currently, it is estimated eight general sizes will be utilized to fit a variety of different sized feet, to accommodate a range of patients. While a carbon fiber or epoxy resin has been utilized in prototype models, it is understood other types of material may be used. In particular, other materials that can be formed such that it will be rigid enough once formed/conformed to patient's foot area. Accordingly, the material used for this invention need not be limited to what is identified herein. In other exemplary embodiments, the use of a 3-D printing process can be employed to make the SCD or can be sourced from a supplier either in bulk “sheets.” It will be appreciated that cassette material 50 may be precut in various shapes and/or sizes using milling, cutting, molding, and so forth, to accommodate various foot shapes and sizes. The thickness of the sheet or form of the underlying cassette material 50 can vary but in prototype embodiments, sample cassette materials 50 having a thickness of 1/32-¼ inches were sampled. Of course, depending on the material composition, its thickness may vary.

FIG. 2B is a top view of a cassette material 50, wherein it has been cut, molded or generated to result in a substantially flat, unfitted SCD first form 60, having “number 2-like” shape, or an hourglass like shape. As noted above, depending on the foot size of the patient, the type of pressure and other modalities wishing to be performed, the size may vary as well as the precise shape shown. Clear to see are three prominent sections of the “unmolded” SCD first form 60, being the bottom plate section 75, appearing trapezoidal to a degree, talus applicator section 95 with a trapezoidal-like shape and a bridging lateral strut section 85 with a linear but curved outline. Various “corners” of the sections are shown with beveled or rounded edges. One end of the talus applicator section 95 can be seen to be greater in length than the other end. The exemplary SCD made in accordance with the present invention is operably configured from the first state, a cassette material primarily flat and “shaped” as seen in FIG. 2B into a second form to fit around the foot of a patient, described in the following FIGS. This second form is understood to be a first “fitting” form which will be customized to the patient's foot to arrive at an operational or final form.

FIG. 2C is a top view of another like-shaped substantially flat, unfitted SCD first form 90. This view is reversal of the orientation of the embodiment seen in FIG. 2B and shows a moderately different sized talus applicator section 95 and a bridging lateral strut section 85. This alternative shaping is indicative of the degrees of freedom available to one of ordinary skill in the art and of the overall compactness of the SCD 60, 90. Of note here is the “smaller” talus applicator section 95 as comparted to in FIG. 2B. Also, the lateral strut section 85 is similar in shape to that of FIG. 2B but slightly thinner. As seen in FIG. 2C the talus applicator section 95 and lateral strut section 85 are of a similar width, thus the talus applicator section 95 can be considered, in some embodiments as an extension of the later strut section 85, wherein a terminal portion is disposed to act as the talus application section 95. It is understood that other variations within the purview of one of ordinary skill in the art are within the scope of this disclosure. In prototype embodiments, for a larger sized SCD 90, the talus applicator section 95 ranged from 1.5-2.5 cm in both width and length, the bottom plate section 75 width ranged from 5-10 cm, and in length from 5-9 cm. The lateral strut section 85 ranged in width of between 1.5-2.5 cm. Of course, depending on the geometry and size of the person's foot, talus, these sizes may vary.

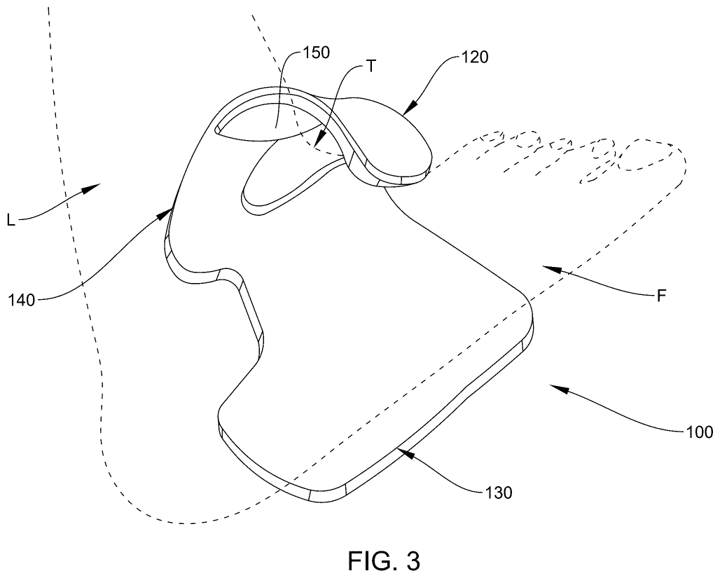

FIG. 3 is an illustration of an exemplary SCD 100 in its second form around a person's foot (phantom-lined). The SCD 100 includes a talus bone pressure applicator (“TPA”) 120. The SCD 100 further includes a base plate (“BP”) 130. The device further includes a lateral strut 140, also referred to as a lateral member (“LM”). The TPA 120, the BP 130 and the LM 140 in this embodiment is shown as a unitary device and together form a main body of the SCD 100. Further, in this particular embodiment, the SCD 100 further includes a Talus Cushion (“TC”) 150.

FIG. 3 also shows a lower leg L and foot F of a user (or patient). The leg L and foot F are shown in phantom lines to illustrate how the device will be worn by the user. In this particular embodiment, the SCD 100 is shown being utilized on the left foot of the user. People have a talus bone disposed near the top of each of their feet. Supination of the foot is when the foot rolls inward toward the outer edge of the foot. The user represented in FIG. 3 has a talus bone area generally referred to as T. It will be appreciated as set forth below that the SCD 100, when it is in a first form (flat) as described in FIGS. 2B-2C, it can be universal and applied to either a right or left foot, if the molding is reversed in orientation. Moreover, in some instances a mirrored version of the SCD can be created for opposite foot use, if so desired.

The SCD 100 is operably configured to apply pressure to the talus bone area T of the user. Specially, the SCD 100 is operably configured and shaped in such a way such that the TPA 120 is directed towards the talus bone area T. Preferably, the TC 150 is disposed adjacent the outer surface of the foot of the user. When the device is in use the TPA 120 is positioned to apply pressure to the talus bone T, and supination of the foot F is substantially inhibited, which is desired. For example, a knowledgeable practitioner would typically apply the appropriate pressure to the talus bone T. Thus, a patient using an SCD 100 with foot supination issues is addressed and the patient can walk substantially with a normal gait. The LM 140 extends up and around the foot F such that the LM 140 is curved over to have the TPA 120 disposed above the talus bone area T of the user when the device is in use. In various embodiments, the TPA 120 is understood to extend a certain distance past the apex of the talus bone to provide a larger surface area for distributed contact on the expanse of the talus bone. In some prototype devices, the TPA 120 extended approximately 1″ past the talus bone apex, noting this distance may vary upon design preference.

The exemplary SCD 100, due to being supportive (rigid) in specific locations while “open” in other locations (providing some degrees of freedom), enables full range-of-motion (“ROM”) for the foot of the user through the user's gait cycle. The gait cycle referring to a sequence of sub-phases, summarized as of stepping forward, putting pressure on the foot, balancing on the foot, weight shifting from rear of foot to the front, ankle/toes rotating for the lifting of the foot, and foot lift. The SCD 100 made in accordance with the present invention is understood to influence several of the sub-phases of the gait cycle.

In some embodiments the SCD will be operably configured to be attached to an existing ankle foot orthosis (“AFO”) device, typically via rivets or other mechanical means. When used in combination with an AFO, foot support or arch support, (not shown) the SCD 100 can be attached or situated onto the AFO/support, wherein the foot F of the user rests on top of the BP 130 or the on top of the AFO/support.

In other embodiments, the SCD is able to be used as a stand-alone unit or as a shoe insert. Also, in other embodiments, a shoe in combination with the SCD may be designed, to act as both a foot supportive device and footwear, being integrated into a single foot appliance. Of course, such designs can be as diverse as the shoe industry (slip-ons, slippers, ankle high, boots, etc.) therefore it is understood one of ordinary skill in the art may utilize whatever means that are available in the shoe arts, as necessary.

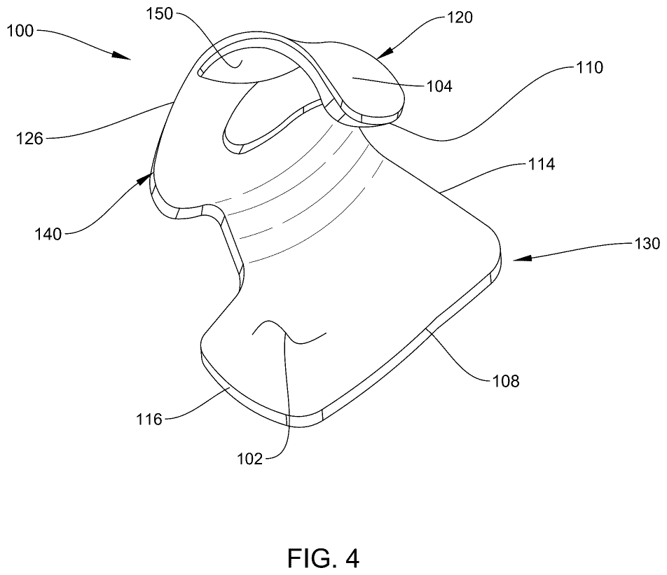

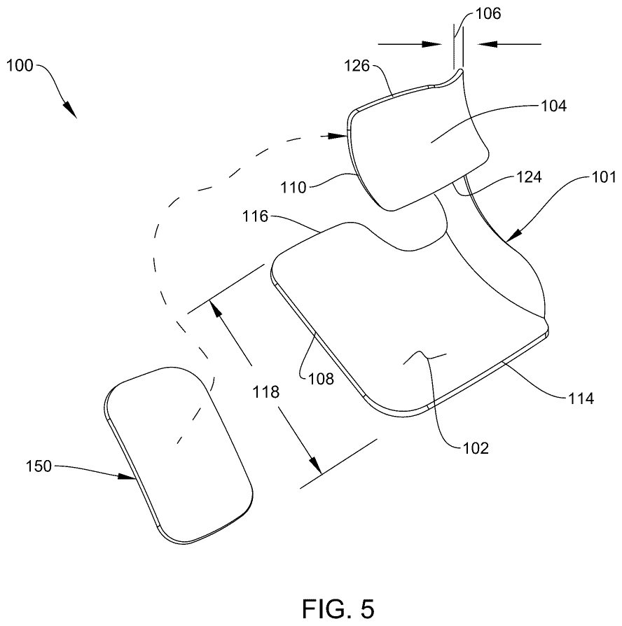

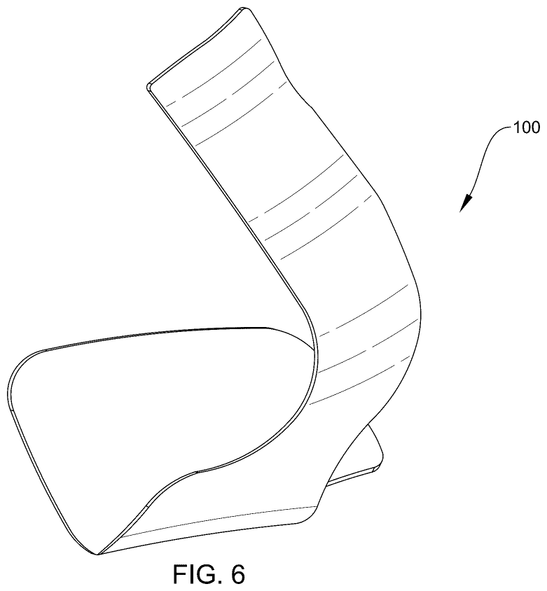

FIGS. 4-9 are different views of an embodiment of an exemplary SCD 100 without a user's phantomed foot and described below. SCD 100 has a main body 101 (called out in FIG. 5), which includes the base plate 102, the TPA 120 and the LM 140 extending between the latter two. In this example the main body 101 is a single unitary body. It should be appreciated that in other embodiments, the SCD 100 can be comprised of multiple individual pieces, joined together.

The LM 130 extends between the BP 140 and the TPA 120, bridging the two via a connection to one side of the BPA 140 and the TPA 120. In this embodiment the LM 130, BP 140 and TPA 120 are integrally connected to each other as a single element. It will be appreciated the LM 130 in other embodiments can be non-unitary with at least one of the BP 140 and the TPA 120. It will be appreciated that the length of the LM 130 can vary, depending on the size of the patient's foot. As seen in the FIGS., the LM 130 is generally narrower in overall width than the BP 140 and TPA 120. At the BP 140 and TPA 120 junctions, the LM 130 increases in width (flares) to provide greater resilience and durability between the various structures. A particularly wide flaring is evident at the lower end of the LM 130—at the BP 140 junction. The overall length of the LM 130 therefore may vary upon the desired flaring and strength requirements, however it is preferably less than the height of the overall SCD 100. The LM 130 will be slightly off-vertical relative to the BP 140 and is typically between 15-20 degrees but may vary depending on the patient's foot and leg geometry and supination objective. Also, the LM 130 can be slightly curved either forward and/or laterally, to allow clearance of the ankle bone and conform to the foot's outer shape. Similarly, the TA 150 is seen to be angled with respect to the BP 140, conforming to a talus bone directed orientation. Depending on the therapy or support being sought, the patient's foot geometry, etc., the TA 150 pressure amount, angle, contact area of the talus bone can be adjusted by a practitioner to allow the patient full range of gait motion.

The unitary or single piece nature of this embodiment of the SCD 100 provides rigidity, ease of attachment, consistency of talus pressure, as well the slim configuration reduces the appearance of its presence (when worn). The thinness of the SCD material provides foot/leg conformity, and specifically the base, enables the SCD to be easily inserted into shoes of the patient. That is, in most cases, the patient need not buy wider shoes, as is common with prior conventional devices. Moreover, because the SCD is preferably a rigid device, having a small degree of flexibility for donning (due to the constituent material), it remains lightweight and more comfortable to wear. Further, the open areas of the SCD allow more freedom than a strap-type device of the conventional type.

The main body of the SCD 100 includes an upper surface 104 in the area of the TPA 120 and a lower surface 102 in the area of the BP 130. A thickness 106 of the SCD 100 is cassette dependent may vary between the first 102 and second surfaces 104. Surface 104 in the area of the TPA 120 is shown to span from leading edge 124 to trailing edge 126, wherein the distance between these edges (124, 126) may vary upon the nature of the patient's foot, as too large an area will cause pinching into the top of the arch and into the bottom of the shin bone. To assist in minimizing potential pinching, a slight curvature can be facilitated on surface 102 as well as to provide some arch conformity in surface 104, if desired.

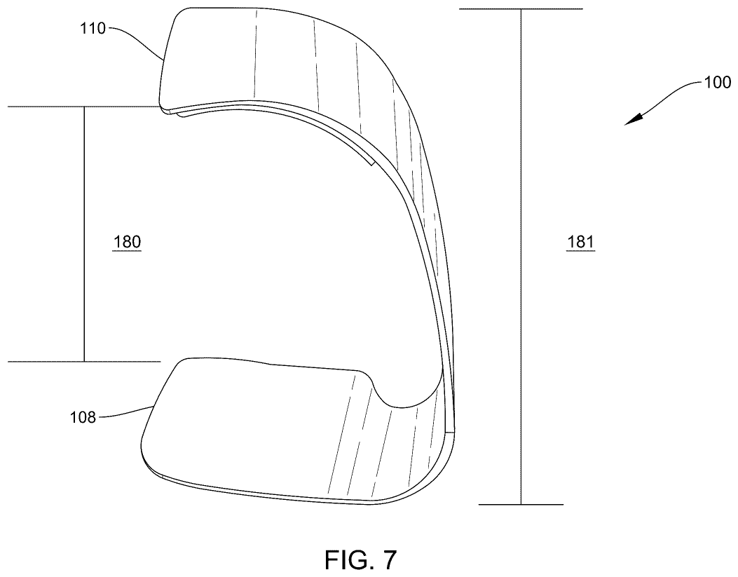

SCD 100 has an upper lateral end 110 in the TPA 120 and a lower lateral end 108 in the BP 130. An SCD length 180 is defined between the upper lateral end 110 and the lower lateral end 108, which bounds the SCD's open side. For clarity, the length 180 is called out in FIG. 7. The “closed” side of the SCD 100 has a height of 181. While the thickness 106 in this embodiment is shown to be substantially uniform throughout, it should be appreciated that the thickness may vary in other embodiments. The BP 130 further includes a leading 114 and a trailing edge 116, opposing the first edge 114. An BPA 130 width 118 is defined between the first 114 and second 116 edges, seen in FIG. 5. The TPA 120 is, in this embodiment, integrated as a unitary element of the upper portion of the SCD 100 bounded by edges, ends 124, 126 and 110.

While not explicitly shown, it will be appreciated that the TPA 120 in other embodiments can be a separate member affixed to the LM 140. Even fitted over the LM 140 being integral and connected to the BP 130. It will be appreciated that also the BP 130 in other embodiments can be a separate member affixed to the LM 140. The BP 130 is preferably substantially planar or flat so it can readily rest comfortably in a shoe and/or be attached to the bottom AFO (not shown). Additionally, in alternative embodiments the TPA 120 and LM 140 may be separately affixed to form the SCD 100, noting such a design will be “integral” upon affixing. The mode of affixing may be permanent in nature, or temporary, according to design preference.

The leading front edge of the TPA 120 forms a curved surface, that is open on one side seen via reference 180. The LM 140 extends from the base plate BP 130 to the TPA 120. The opening 180 is approximately 40-80% of the overall height 181 of the SCD. It is preferred that the opening be about 50-70% of the overall height 181. It is more preferred that the opening be about 60% of the overall height 181. Depending on the foot size, the 180 opening can range between 2-6 inches and the 181 length can similarly range between 2-6 inches.

FIG. 5 illustrates the exemplary SCD 100 without an attached talus cushion (TC) 150. Accordingly, in various exemplary embodiments the SCD does not need to include a cushion, it is optional based on the desired level of pressure to be applied and comfort for a user. Typically, but not necessarily, the desired pressure can be between 2-3 lbs. In FIG. 5, the TC 150 is removed, and shown set aside, from the TPA 120 for purposes of identifying other features and elements of the SCD 100. The TC 150 can be made from a foam material and preferable a closed cell foam material. In a prototype embodiment the cushion was formed from Pelite. When utilized in an SCD 100, the TC 150 is disposed on a portion of the underside of surface 104 of the TPA 120. The TC 150 may be affixed to the TPA 120 via a fastener. The fastener can be an adhesive either separate to or part of the TC 150. In some embodiments, the adhesion method may be via a mechanical means, for example, rivets, a sleeve, hook/loop fastening, etc. It will be appreciated that the fastener can be other conventional alternatives such as double-sided tape and the like. It will be appreciated that additional cushions or other padding may be integrated into the SCD 100 if desired. As well as having the TC 150 take the form of a wrapping or like.

It will be appreciated that the BP 130 can be rigid prior to fitting and attached to an AFO (not shown) via rivets, for example, or inserted into shoes and held in place by rivets, screws, hook and loop fasteners, adhesive tape, glue or any other conventional fastening means.

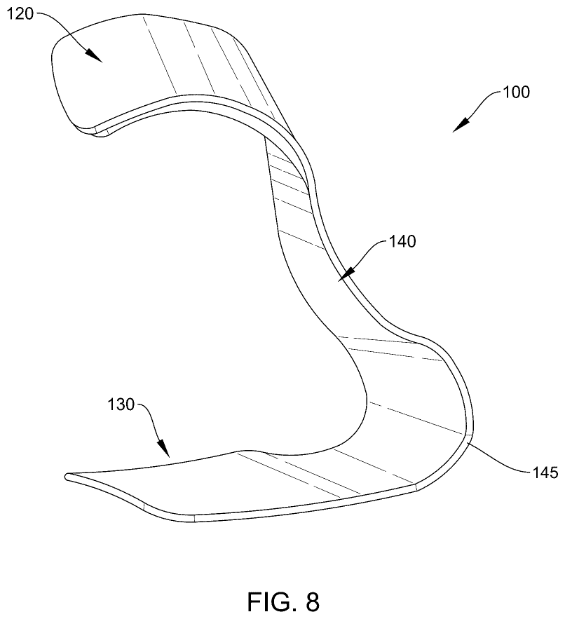

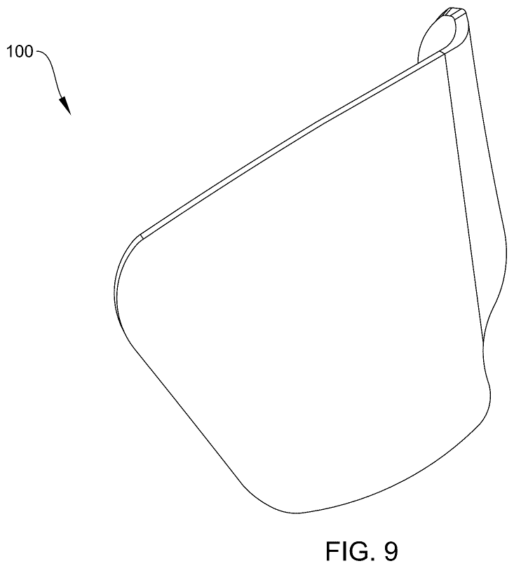

FIGS. 8 and 9 serve to illustrate the various curvatures noted above representative of an embodiment of an exemplary SCD 100, wherein FIG. 8 is a frontal view and FIG. 9 is a bottom view. In this embodiment, the cupping, curved shape of the TPA 120 is evident, noting the degree of curvature can be patient-dependent. Moreover, depending on the cushioning (TC) utilized, its size, density, shape, the TPA's “flatness” may vary. That is, a thick rounded cushion TC may require a different curvature in the TPA 120. FIG. 8 shows a curvature and angle of the LM 140 with its bowing outward when joined to the BP 130. This contour or undulation of the LM 140's shape, in combination with the constituent material of the SCD 100, provides a compact structure which allows a slight degree of flexing of the LM 140, yet provides sufficient rigidity to support the TPA 120. FIG. 9 shows a slight difference in the width-to-length of the BP 130, which corresponds to the natural widening in a person's foot.

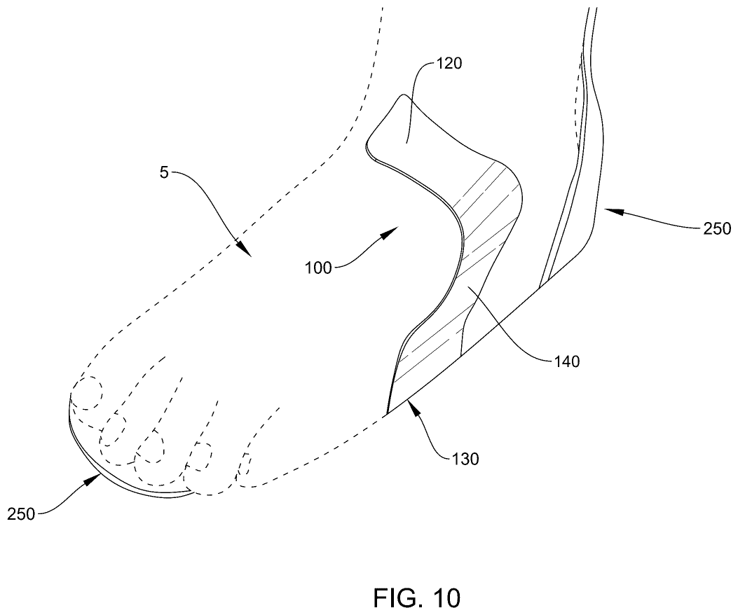

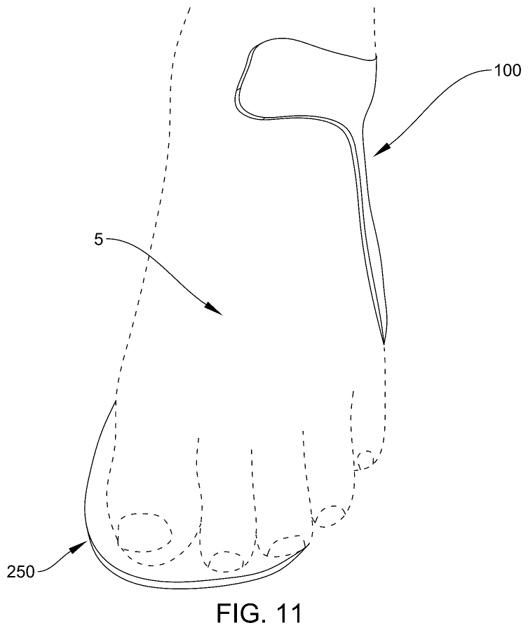

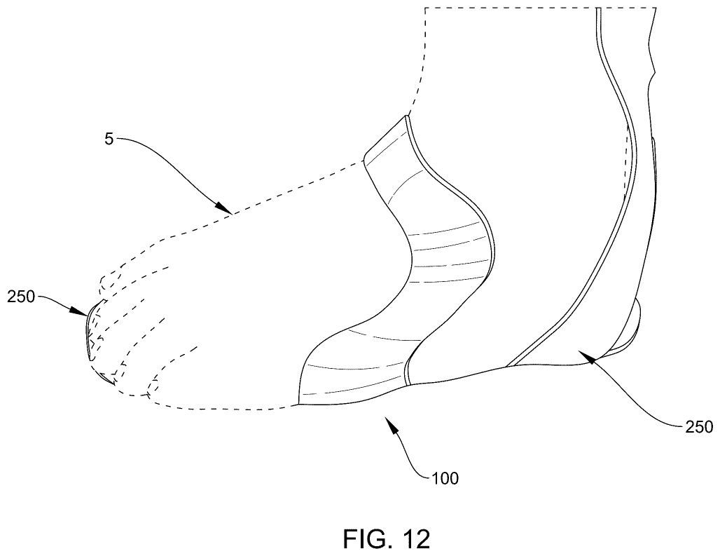

FIGS. 10-12 illustrate various views of an exemplary SCD 100 being utilized on a foot 5 of a person also wearing an AFO. The SCD 100 is shown as used on the left foot 5 of the person. In this example, the SCD 100 is affixed to a conventional AFO 250 having a sole length substrate. While no shoe or other footwear is worn in this example, it is understood that some kind of footwear may be worn over the SCD 100 (and AFO 250). It will be appreciated as illustrated in these figures, SCD 100 is operably configured such that when the device is in use, it enables the TPA 120 to be disposed adjacent the talus bone area of the person. The foot 5, when the SCD 100 is in use, is disposed on top of the BP 130, which is hidden in these figures by the foot 5.

As the SCD 100 is applied to a human's foot 5, some accommodation for fit and comfort as well as supination-correction purposes are to be expected. Thus, a fitting process is applied, transforming the second form of the SCD 100 as shown in FIGS. 3-9, and then into a “fitted” operational form, as demonstrated in FIGS. 10-12. In the second unfitted form, the TPA 120 and the LM 140 may be loose—that is, the needed pressure/support of those structures to the foot 5 may be lacking. This looseness may be a factor of the cassette material used, such that the TPA 120 and LM 140 may either be semi-rigid, flaccid. Alternatively, the TPA 120 and LM 140 may be fully rigid, but non-conformal to the desired final shape. It is understood the second form of the SCD 100 is where the SCD 100, when fitted around a user is such that the TPA 120 is properly or almost properly positioned.

After second form alignment, the final operational form is acquired by adjusting the TPA 120 and/or the LM 140 relative to the patient and hardened into its appropriate position. The hardening of the SCD 100 can be performed by applying UV light, with the understanding that for this example, a cassette material and/or resin is used that is sensitive to UV light. A UV light hardening process is particularly advantageous due to its quick “hardening” effect on UV sensitive materials. Generally, the cassette material acts as a flexible substrate wherein a solution or gel, having a UV sensitivity, is applied and then UV hardened. Conventional carbon fiber or mesh materials and their hardening process (resins, etc.) can also provide the desired results. Epoxies are also capable of rapid hardening when mixed. Other types of materials can be hardened by a process of heating and cooling, wherein the heating allows the material to flex and moved into desired shape and upon cooling, the material returns to its original rigidity. Various materials of this and the above calibers are well known in the manufacturing arts and incorporated herein.

In prototypes utilized for this invention, a cassette material of raw carbon braid was tested and an epoxy and acrylic mixed hardening resin was applied. Depending on the resin type used, the curing (hardening) could come inherently from the “mix” or applying a UV light for a resin-dependent period of time. In some instances the resin would harden within minutes. As stated above, alternative approaches are available to one of ordinary skill in the art.

In another prototype, a heating approach was tested, wherein the cassette was of a material which became flexible after being heated to a desired temperature. For the material used (a no-brand carbon braid, polypropylene braid) the cassette was heated to about 300 degrees Fahrenheit for about 3 minutes, noting the temperature and time may vary upon the material composition. And while in the flexible state, after final adjustment the cassette material cooled to a solid, rigid state. This process could take less than ten minutes.

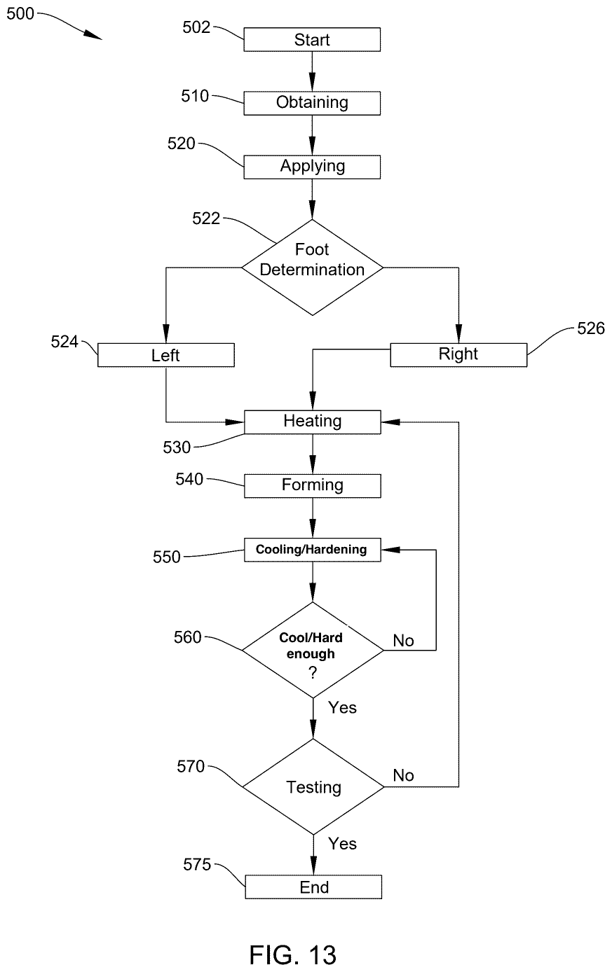

FIG. 13 shows steps of an exemplary forming method 500, in accordance with the present invention. This method will be utilized by a person or a practitioner that is fitting the device on another person or patient. It will be appreciated that the fitter may be a licensed certified orthotist. It will be appreciated that more than one person or machine may assist with various steps as detailed herein.

The exemplary method 500 begins at an obtaining step 510, wherein a cassette of material suitable for an SCD is gathered and converted into a planar “shaped” first form, using any number of ways as described above, having an appearance similar to that seen in FIGS. 2B, 2C. For the purposes of this example, the cassette is presumed to be thermally deformable. It is understood however, a non-thermal responsive cassette material may be used, as discussed above. Implicit in this step 510 is an awareness of the patient's foot size so as to obtain a matching size in the first form. This awareness of the patient's foot size may be independent of this obtaining step 510 and may occur prior to, or be a part of the following step. It is understood that foot size determination is typically performed by measuring the length needed to encapsulate the talus bone of the patient, typically by first measuring from a bottom of the foot (and appliance if used) to approximately an inch within or near the apex of the talus bone. Once a measurement is obtained, a corresponding cassette is chosen having a length at least as long as the obtained measurement.

The exemplary method 500 proceeds to step 520 wherein a thermal sock is placed on the patient or person's foot. It is preferred that the thermal sock be made of any conventional heat resistive flexible materials, such as Kevlar. A thermal sock will help prevent burning of a patient's skin when thermally forming the SCD into its second upright form and operation form. If the cassette material is of a non-thermally deformable material such that a resin or UV is the means for hardening/stiffening, then this step 520 may be skipped.

The exemplary method 500 then moves to a foot determination step 522. In this step, it will be determined to which foot the SCD will be applied. If it is a left foot, the method proceeds to step 524, wherein the fitter will choose a surface (top or bottom) of the cassette to position the cassette such that it will be properly oriented to the foot of the patient when in use. Otherwise, the method proceeds to step 526, and the opposite foot will dictate the surface (bottom or top) of cassette will be used to for the foot of the patient. In other words, the cassette is flipped over depending on which foot (right or left) is being treated.

The exemplary method 500, when using a thermally conformal material, includes a heating step 530. It is preferred to heat the LM and the TPA material to a near-to-phase-altering state, which is about 300 degrees Fahrenheit for about 3 minutes or so in order for those members to become easily hand-pressure deformable due to its heat-induced pliably. This step 530 can be accomplished by placing an appropriately sized cassette into a heated oven and after a period of time (allowing the heat to soften the cassette material), remove it from the oven.

The exemplary method 500 proceeds to a forming step 540, wherein while the LM and TPA remain pliable, they are bent wherein the TPA formed over the talus bone of the patient, such that the BP is also positioned underneath the surface of the patient's foot/appliance. In some instances, it may not be necessary to heat the LM as it may be sufficiently positioned. The forming step 540 is to allow a desired pressure to be applied to the patient's talus bone, which can be in the range of 2-3 lbs, which may vary beyond these values, depending on the practitioner's discretion. A cushion may be added in this step 540 or in the subsequent hardening/testing steps to obtain a desired pressure level using different thicknesses, density cushions, etc.

The exemplary method 500 further includes a hardening step 550 which is accomplished by allowing the TPA to harden while in place. This step 550, when using a heating approach, permits the heated TPA (and/or any other part of the SCD) to cool down. Generally, this may take less than ten minutes or faster.

The exemplary method 500 proceeds with a determining step 560, wherein it is determined if the SCD is hard enough, typically by feeling if the cassette material is cool to the touch, or the formed sections are rigid when pressed. If not hardened, the method 500 repeats cooling/hardening step 550. If yes, the exemplary method 500 can optionally have the practitioner attach a pad to the underside of the TPA to compensate for any excess separation as well as adjust for a correct amount of pressure.

The exemplary method 500 continues to a testing step 570. If the user is wearing (or intending to wear) an AFO or a shoe or other type foot application, it is preferred that the testing step 570 include fitting the SCD to that user's foot appliance and having user walk with the device. If the testing step 570 does not prove satisfactory, the exemplary method 500 goes back to the heating step 530 to redo the heating (in whole or in part) and subsequent fitting steps to the user. If yes, the exemplary method 500 ends.

It is understood that while the above exemplary method 500 is described for a heating process, it may be modified in accordance with alternative material and forming characteristics, as described above. It is noted that utilizing a heating approach provide an adjustment/testing option that is not generally available with a resin approach, as once the resin has hardened, testing and “re-softening” can be more complicated than with a heating approach. Nonetheless, either or other approaches are available to a practitioner.

The previous description of the disclosed embodiments and methods is provided to enable any person skilled in the art to make or use the present disclosure. Various modifications to these embodiments will be readily apparent to those skilled in the art, and the generic principles defined herein may be applied to other embodiments without departing from the spirit or scope of the disclosure. Thus, the present disclosure is not intended to be limited to the embodiments shown herein but is to be accorded the widest scope consistent with the principles and novel features disclosed herein.

What is claimed is:

1. An open sided foot supination control device, comprising:
a base plate having a first end and an opposing second end;
a talus bone pressure applicator having a first end and an opposing second end; and
a lateral member having a first end and second end, a first end of the lateral member joined to the first end of the base plate, and a second end of the lateral member joined to the first end of the talus bone pressure applicator.

2. The supination control device of claim 1, wherein the base plate, talus bone pressure applicator and lateral member are unitary, coplanar to each other and formed from a sheet of material, the base plate being larger than the talus bone pressure applicator and the lateral member forming a curved connecting structure between the base plate and the talus bone pressure applicator.

3. The supination control device of claim 1, wherein the base plate, talus bone pressure applicator and lateral member are in an operational form, wherein the base plate is configured to be disposed on a bottom of a foot and the talus bone pressure applicator is configured to be disposed over a talus bone of the foot.

4. The supination control device of claim 3, further comprising a cushion disposed on an underside of the talus bone pressure applicator.

5. The supination control device of claim 4, wherein at least one of the talus bone pressure applicator and cushion is configured to actively apply pressure to a talus bone of a foot.

6. The supination control device of claim 1, wherein a shape of the talus bone pressure applicator is curved.

7. The supination control device of claim 1, wherein an overall shape of the foot supination control device appears to represent a number “2.”

8. The supination control device of claim 2, wherein a thickness of the sheet of material is between 1/32 to ¼ inches.

9. The supination control device of claim 8, wherein the sheet of material is composed of at least one of a carbon fiber, polypropylene, nylon, and plastic.

10. The supination control device of claim 8, wherein the sheet of material becomes temporarily moldable upon an application of heat.

11. The supination control device of claim 8, wherein the sheet of material becomes hardened upon an application of at least one of a resin and UV light.

12. The supination control device of claim 3, wherein a height of the supination control device is between 3-6 inches.

13. A foot supination control device, comprising:
a base plate having a first end and an opposing second end, the base plate configured to configured to be disposed on a bottom of a foot;
a talus bone pressure applicator having a first end and an opposing second end, disposed above the base plate and having a size smaller than the base plate; and
a lateral member having a first end and second end, a first end of the lateral member joined to the first end of the base plate, and a second end of the lateral member joined to the first end of the talus bone pressure applicator, wherein the lateral member is configured to rise from the base plate to the talus bone pressure applicator and conform to an outer shape of the foot,
wherein the talus bone pressure applicator is configured to actively apply pressure to a talus bone of the foot and the foot supination control device provides unrestricted gait motion.

14. The supination control device of claim 13, wherein the base plate, talus bone pressure applicator and lateral member are unitary and formed from a sheet of material, the base plate being larger than the talus bone pressure applicator and the lateral member forming a curved connecting structure between the base plate and the talus bone pressure applicator.

15. The supination control device of claim 13, further comprising a talus cushion disposed on an underside of the talus bone pressure applicator.

16. The supination control device of claim 13, further comprising an Ankle Foot Orthosis (AFO) device, the supination control device being coupled to the AFO.

17. The supination control device of claim 13, wherein the applied pressure is between 2-3 lbs.

18. A method of fitting a supination control device to a foot of a user, comprising:
measuring a size of a user's foot;
gathering a rigid substrate material, having a base plate, a talus bone pressure applicator and a lateral member, corresponding to the measured foot size;
altering the rigid substrate material into a softened state by at least one of heating and applying a softening agent to the substrate material;
fitting a protective sock over the user's foot;
placing the base plate under the user's foot and bending the lateral member and talus bone pressure applicator to position the talus bone pressure applicator adjacent to a talus bone of the user, while the substrate material is in the softened state;
obtaining a talus bone pressure of between 2-3 lbs via at least one of pressing the talus bone pressure applicator and securing a pressure-inducing cushion to the talus bone pressure applicator; and
hardening the softened substrate material into a rigid state by at least one of cooling, chemical treatment and UV light exposure to the softened substrate material.

19. The method of claim 18, further comprising, testing a fit of the supination control device.

20. The method of claim 18, wherein the protective sock is a thermally resistant sock.

21. The method of claim 18, further comprising forming a first planar state of the supination control device from a sheet of material, by at least one of milling, cutting, molding, and 3-D printing the sheet of material to form the base plate, a talus bone pressure applicator and a lateral member.

22. The method of claim 21, wherein a shape of the first planar state of the supination control device has an appearance of a numeral “2.”
